(19) United States
(12) Patent Application Publication
(10) Pub. No.: US 2010/0184871 A1
(43) Pub. Date: Jul. 22, 2010

(54) Polyglycerol Derivatives

(76) Inventors: Hanamanthsa Shankarsa Bevinakatti, Cleveland (GB); Alan Geoffrey Waite, County Durham (GB); Jackie Frank, Cleveland (GB)

Correspondence Address: JONES DAY, 222 EAST 41ST ST, NEW YORK, NY, 10017, US

(21) Appl. No.: 12/452,951

(22) PCT Filed: Jul. 30, 2008
(86) PCT No.: PCT/GB2008/002602

§ 371 Date: Mar. 9, 2010

(30) Foreign Application Priority Data
Jul. 31, 2007 (GB) 0714817.4

(51) Int. Cl. C07D 307/04; A61K 8/06; A23D 7/00; C09K 3/00; B01F 17/32; C10M 115/04

(52) U.S. Cl. 514/785; 549/501; 426/602; 252/194; 516/74; 508/308

(57) ABSTRACT

Polyglycerol ethers of sorbitan carboxylic acid, particularly C₈ to C₂₂ carboxylic acid, esters are new surfactant compounds, useful as emulsifiers. Desirable compounds are of the formula (I): Sor(R¹)(R²)(R³)(R⁴) where R¹, R², R³, R⁴ have defined meanings such that at least one group is of the formula (II): -G₂CR⁵, where R⁵ is a C₇ to C₂₁ hydrocarbyl group, and at least one is of the formula (III): -[Gly]-[AO]ₘ-H where Gly is a glycerol residue, AO is an alkyleneoxy residue of a corresponding diol cyclic carbonate, in any order; n is an average of from 0 to 100; and m is an average of from 0 to 75; such that the total of all the indices n is at least 1.

This invention relates to polyglycerol ethers of sorbitan carboxylic acid esters, particularly esters with relatively long chain fatty acids, to their manufacture and use as surfactants, particularly emulsifiers.

Sorbitan is a C₆ compound which is a C₄ cyclic ether generally with a 2-carbon side chain. It is generally the product of the dehydration of sorbitol, usually by thermal dehydration under acid catalysis. In practice sorbitan is a mixture of isomers principally 1,4-anhydro-D-glucitol [1-(1,2-di-hydroxy)ethyl-2,3-dihydroxytetrahydrofuran), but may include 2,5-anhydro-D-glucitol (1,4-di-(hydroxymethyl)-2,3-dihydroxytetrahydrofuran), 1,5-anhydro-D-glucitol (1-hydroxymethyl-2,3,4-tri-hydroxytetrahydropyran) and may include di-cyclic diethers such as iso-sorbide as impurities. For convenience, where sorbitan is referred to herein as a single compound it will be understood that this is a simplification in that sorbitan, and sorbitan residues in esters and derivatives, is almost invariably a mixture of various cyclic ethers or their residues and such reference includes the various mixtures of isomers in typical sorbitan.

Although sorbitan is a known compound and can be obtained as such, it is most usually commercially found as a component of surfactants, particularly sorbitan esters, available under the Trademark “Span” from Croda Europe Ltd (“Croda”) and their polyethoxylated derivatives available under the Trademark “Tween” (from Croda). Surfactant sorbitan esters are most usually made by reaction of sorbitol with a fatty acid using a mildly acidic catalyst. Sorbitan esters are attractive and widely used relatively hydrophobic surfactants e.g. as water in oil emulsifiers, which can be made from sustainably sourced raw materials—principally fatty acids (from natural fats or oils) and sorbitol (from hydrogenation of glucose). Commercially, polyethoxylated sorbitan esters typically have ten or more ethyleneoxy (EO) residues for each sorbitan residue and are thus much more hydrophilic and find widespread use e.g. as oil in water emulsifiers. Because polyethoxylated sorbitan esters include significant proportions of EO residues they are nowadays increasingly seen as less “sustainable” than sorbitan esters, because of the use of petrochemically derived ethylene oxide in their manufacture.

Glycerol has been proposed as an alternative source of hydrophilicity to EO in surfactants. To date it has not been perceived as being particularly successful in this role, although glycerol based surfactants e.g. fatty acid polyglycerol esters, have found niche applications.

Glycerol/glycerine carbonate (4-hydroxymethyl-1,3-dioxolan-2-one) has been known as a compound for many years. It has become commercially available from routes including reacting glycerol with phosgene or an alkyl(ene)carbonate, see U.S. Pat. No. 2,915,529 or JP 63-029663 A, catalytic reaction of glycerol, carbon monoxide and oxygen, see U.S. Pat. No. 5,359,094, or reacting urea with dialkyl carbonates, see U.S. Pat. No. 6,025,504 or U.S. Pat. No. 6,495,703. Prior described reactions with glycerol carbonate generally utilise reagents which are miscible with glycerol carbonate e.g. glycerol to make polyglycerol—see U.S. Pat. No. 5,721,305, U.S. Pat. No. 5,723,696, JP 10-072392 A and JP 10-072393 A, or other short chain polyols such as trimethylol propane to make hyperbranched polyethers—see G. Rokicki et al, Green Chemistry, 2005, 7, 529.

This invention is based on our discovery that polyglycerol analogues of polyethoxylated sorbitan carboxylic acid esters can be made having properties similar to those of the polyethoxylated sorbitan carboxylic acid esters. The polyglycerol analogues can be made by reacting sorbitan carboxylic acid esters with glycerol carbonate.

The present invention accordingly provides a compound which is a polyglycerol ether of a sorbitan carboxylic acid ester, particularly a fatty, especially a C₈ to C₂₂, carboxylic acid sorbitan ester, particularly a monoester.

Alternatively, the invention may be described as including compounds obtainable by the reaction of a sorbitan ester with glycerol carbonate, desirably at least 1 and particularly at least 3 moles of glycerol carbonate per mole of sorbitan ester.

In particular, the compounds of the invention are of the formula (I):

Sor(R¹)(R²)(R³)(R⁴) (I)

where
Sor is a sorbitan residue;
   one of R¹, R², R³ and R⁴ is a group of the formula (II): —O₂CR⁵ where R⁵ is a C₇ to C₂₁ hydrocarbyl group;
one of R¹, R², R³ and R⁴ is a group of the formula (III): -[Gly]ₙ[AO]ₘ-H where Gly is a glycerol residue; AO is an alkyleneoxy residue of a corresponding diol cyclic carbonate, in any order;
   n is an average of from 0 to 100; and m is an average of from 0 to 75;
each of the remaining two of R¹, R², R³ and R⁴ is independently:
   a group of the formula (IIa): —O₂CR^{5′} where R^{5′} is a C₁ to C₂₁ hydrocarbyl group; or
   a group of the formula (III): -[Gly]ₙ-[AO]ₘ-H where each Gly, AO, n and m is independently as defined above;
such that the total of all the indices n is at least 1.

The invention includes a method of making compounds of the invention, which comprises reacting a sorbitan ester with at least 1 and desirably at least 3 moles of glycerol carbonate per mole of sorbitan ester.

The invention includes a method of making compounds of the formula (I), which comprises reacting a sorbitan ester of the formula (IV):

Sor(R^{1′})(R^{2′})(R^{3′})(R^{4′}) (IV)

where
Sor is a sorbitan residue;
one of R^{1′}, R^{2′}, R^{3′} and R^{4′} is a group of the formula (II): —O₂CR⁵ where R⁵ is a C₇ to C₂₁ hydrocarbyl group;
one of R^{1′}, R^{2′}, R^{3′} and R^{4′} is a hydroxyl group; and
each of the remaining two of R^{1′}, R^{2′}, R^{3′} and R^{4′} is independently a hydroxyl group, or a group of the formula (IIa): —O₂CR^{5′} where R^{5′} is a C₁ to C₂₁ hydrocarbyl group;
with at least 1, and desirably at least 3, moles of glycerol carbonate per mole of sorbitan ester of the formula (IV).

In the formulae (I) and (IV) above, the group “Sor” is a sorbitan residue i.e. after removal of 4 hydroxyl groups from sorbitan, and typically is the residue of 1,4 anhydro-D-glucitol; 2,5-anhydro-D-glucitol; or 1,5-anhydro-D-glucitol and in practice will usually be a mixture of such isomers, often in practice also including iso-sorbide as an impurity.

In the compounds of the invention, the acid used to make the sorbitan ester which is the basis of the polyglycerol ether will generally be a monocarboxylic acid in which the carboxylic acid residue is of a relatively long chain carboxylic acid. Di- or tri-carboxylic acid sorbitan ester may be used as the basis of the polyglycerol ethers, but such sorbitan di- or tri-esters will be significantly more hydrophobic and provide fewer hydroxyl reaction sites than monoesters and are thus less preferred.

Thus, with reference to formula (I), in desirable compounds of the invention one of the groups R¹, R², R³ and R⁴ is a group of the formula (II): —O₂CR⁵ and the remaining three groups are of the formula (III): -[Gly]ₙ-[AO]ₘ-H where R⁵, Gly, n and m are as defined above.

The carboxylic acid residue(s) in the sorbitan ester [corresponding to the residue —O₂CR^{5′} in formulae (IV) and (I)] may broadly be of C₂ to C₂₂, typically C₆ to C₂₂, carboxylic acids. As the products will commonly be used as surfactants at least one of the acid residue(s) is, and more usually all will be (though most commonly there will be just one) of C₈ to C₂₂, typically C₁₀ to C₂₂, and particularly C₁₂ to C₁₈, monocarboxylic acids [corresponding to the groups —O₂CR⁵ in formulae (IV) and (I)]. The carboxylic acid residue(s) may be of linear or branched, saturated or unsaturated acids, and suitable examples include residues of lauric, myristic, palmitic, palmitoleic, stearic, iso-stearic (a mixture of mainly branched acids with a range of chain lengths averaging about C₁₈), oleic, linoleic, linolenic, behenic, erucic or omega 3-, 6- or 9-fatty, acids. Mixtures of residues of carboxylic acids may be used if desired.

The inclusion of shorter chain carboxylic acid residues e.g. C₂ to C₇ residues, is possible, but will require an additional process step which is likely to make such products not cost competitive with monocarboxylic ester derivatives or di- or tri-ester derivatives where the carboxylate residues are all relatively long chain residues.

Glycerol is incorporated into the compounds of the invention as glycerol residues, corresponding to the group “Gly” in formula (I). These can be considered as divalent residues of a corresponding diol, of one of the formulae, —OCH₂CH(CH₂OH)— or —OCH₂CHOHCH₂— or, where the chain branches, a trivalent residue of the formula —OCH[CH₂O—]₂. Where the chains are at least two glycerol residues long, it is further possible that cyclic diglycerol units may be formed. The presence of cyclic diglycerol units is not particularly desirable because their formation reduces the number of hydroxyl groups along the chain thus making the chains less hydrophilic.

Generally the glycerol ether units in the compounds of the invention are homopolymeric polyglycerol chains—corresponding to the total of the indices m being 0. However, if desired, other divalent diol residues, particularly those derivable from cyclic carbonates other than glycerol carbonate, may be included—corresponding to the total of the indices m being greater than 0, usually at least 0.1. Examples of such diol residues include ethyleneoxy, 1,2-propyleneoxy and 1,3-propylene-oxy; ethyleneoxy and 1,2-propyleneoxy residues being familiar to surfactant chemists from products made using the corresponding alkylene oxides. Such inclusions will modify chain properties somewhat, in particular with 1,2- and 1,3-propyleneoxy units tending to make the chains less hydrophilic. The proportion of such other chain residues used will typically be less than 75, more usually less than 50 and generally less than 25, mole % of the total diol residues used in the synthesis. Where combinations of glycerol and other diol residues are included in compounds of the invention, the copolymeric chains may be random (statistical) or block, including taper block, sequential block, block random and similar types of copolymeric chains.

The copolymeric types of polyethers of sorbitan esters described above are compounds of the invention and the invention accordingly includes a mixed poly(alkyleneoxy)/polyglycerol ether of a sorbitan carboxylic acid ester, particularly a fatty, especially a C₈ to C₂₂, carboxylic acid.

In particular the mixed esters are of the formula (Ia):

Sor(R^{1a})(R^{2a})(R^{3a})(R^{4a}) (Ia)

where
Sor is a sorbitan residue;
one of R^{1a}, R^{2a}, R^{3a} and R^{4a} is a group of the formula (II): —O₂CR⁵ where R⁵ is a C₇ to C₂₁ hydrocarbyl group;
one of R^{1a}, R^{2a}, R^{3a} and R^{4a} is a group of the formula (IIIa′): -[Gly]_{n′}[AO]_{m′}-H where Gly is a glycerol residue and AO is an alkyleneoxy residue of a corresponding diol cyclic carbonate, in any order; n′ is an average of from 0 to 100; and m′ is an average of from 0 to 75;
each of the remaining two of R^{1a}, R^{2a}, R^{3a} and R^{4a} is independently:
   a group of the formula (IIa): —O₂CR^{5′} where R^{5′} is a C₁ to C₂₁ hydrocarbyl group; or
   a group of the formula (Mal: -[Gly]_{n′}[AO]_{m′}-H where Gly n′ and m′ are as defined above; such that the total of all indices n′ is at least 1 and the total of all indices m′ is at least 0.1, with typically the total of all the indices n′ and m′ being at least 2, and usually for each chain independently n′+m′ is from 2 to about 100.

The invention further includes a method of making a mixed poly(alkyleneoxy)/polyglycerol ether of a sorbitan carboxylic acid ester which comprises reacting a sorbitan ester with at least 3 moles of a combination of glycerol carbonate and a cyclic carbonate of ethylene glycol, propylene glycol or 1,3-propylene diol, per mole of sorbitan ester.

Typically, the average degree of polymerisation (DP) [corresponding to the total of the indices n or n+m in formula (I) and n′+m′ in formula (Ia) respectively] of the compounds of the invention will be from 1 to 100, more usually 5 to 75 and particularly from 10 to 50, and the chain length, of individual chain(s) [corresponding to the average value of n or n+m in formula (I) and n′+m′ in formula (Ia) respectively] will be in the range from 1 to about 40, particularly from 2 to 20, and commonly at least one chain will be at least 3 residues long.

The compounds of the invention are generally mixture of (poly)glycerol ethers of sorbitan esters having a range of DP and (individual) chain length.

The compounds of the invention, can be made by reacting a sorbitan ester with glycerol carbonate. Typically, the molar ratio of sorbitan ester to glycerol carbonate used in the synthesis is generally at least 1:1, more usually from 1:2 to 1:100, typically 1:3 to 1:75, though more usually from 1:3 to 1:50, desirably 1:3 to 1:40 and particularly from 1:3 to 1:30. Although the synthetic reaction appears robust enough to make products with average degree of polymerisation (DP) greater than about 30, reaction rates may fall off somewhat at higher DP values, which may be compensated for by top up (or continuous) addition of glycerol carbonate and/or catalyst. At such high DPs the synthetic reaction will generally take longer giving more time for side reactions thus giving rise to lower product purity (see below).

Where the sorbitan ester and glycerol carbonate are immiscible, at the start of the reaction, the reactants form a two phase liquid system. As the (poly)glycerol chain of the etherified esters grows, the polyethers become increasingly miscible with glycerol carbonate. Thus, the products and to an extent the intermediate ethers will tend to act to compatibilise the starting materials, but when the transition to a single phase system occurs will depend on the reagents used. Reaction between components (generally) in different phases will be slower than when they are in one phase. The degree of compatibility of the intermediate esters may influence the relative speed of reaction as against chain length and thus influence the spread of chain lengths in the final product. If desired, the physical immiscibility of the starting materials may be avoided by the use of suitable solvent(s) (see below).

The reaction proceeds slowly unless a catalyst, particularly a base catalyst, is used, and the invention accordingly includes a method of making a polyglycerol ether of a sorbitan carboxylic acid ester in which a sorbitan ester is reacted with glycerol carbonate, in the presence of a base catalyst. Without being bound by any particular explanation, we believe the catalyst reacts with free OH group(s) on the sorbitan ester to form alkoxide ions which react with the carbonate by a nucleophilic reaction, displacing the carbonate at the 1- or 2-position in the glycerol, with subsequent decarboxylation with evolution of CO₂. Subsequent chain extension reaction steps appear to involve base reacting with free OH on the intermediate etherified sorbitan ester to form alkoxide (in the early stages of the overall reaction either on the sorbitan ester or on glycerol units etherified directly or indirectly to it) which reacts further with carbonate analogously. Suitable catalysts include alkali metal, particularly sodium or potassium, bases e.g. hydroxides, particularly NaOH or KOH, carbonates, particularly K₂CO₃ or Na₂CO₃, bicarbonates, particularly KHCO₃ or NaHCO₃ and alkoxides particularly sodium or potassium lower, particularly C₁ to C₄, alkoxides e.g. sodium or potassium methoxide, and tertiary amines, particularly tertiary amines including at least one tertiary nitrogen atom in a ring system, such as 1,8-diazabicyclo[5.4.0]undec-7-ene (DBU), 1,4-diazabicyclo[2.2.2]octane (DABCO), 4-(dimethylamino)pyridine (DMAP), 7-methyl-1.5.7-triazabicyclo[4.4.0]dec-5-ene (MTBD), quinuclidine, pyrrocoline and similar materials. Base catalyst, particularly alkali metal hydroxide may be partially neutralised (or buffered) with acid, particularly fatty acid used in the esterification reaction—in effect using a fatty acid soap as catalyst—or a polybasic acid such as phosphorus oxyacid e.g. phosphoric acid, or (see also below) reducing phosphorus oxyacids such as phosphorous acid.

The amount of catalyst used will typically be from 0.5 to 25, more usually 2 to 20, and particularly 5 to 15, mol %, based on the sorbitan ester starting material. Potassium carbonate, desirably used in an amount of from 3 to 18, especially from 5 to 15 mol % based on the sorbitan ester starting material, is a particularly useful catalyst.

Particularly when catalysed, we have found that the reaction proceeds readily to completion i.e. complete consumption of the glycerol carbonate. This gives rise to a practical benefit of the invention that the molar ratio of sorbitan ester starting material to glycerol carbonate used generally determines the (average) number of glycerol residues in the product (but see below on side reactions).

To make the copolymeric compounds of the invention e.g. of the formula (Ia), the synthesis will typically be carried out using other cyclic carbonates e.g. ethylene glycol, propylene glycol and/or propylene-1,3-diol (trimethylene)carbonate, in addition to glycerol carbonate. The proportion of such other carbonates used will be chosen to provide the corresponding level of copolymeric inclusion in the chains and accordingly will typically be less than 75, more usually less than 50 and generally less than 25, mole % of the total carbonate used in the synthesis. The invention further includes a method of making a mixed poly(alkyleneoxy)/polyglycerol ether of a sorbitan carboxylic acid ester in which a sorbitan carboxylic acid ester is reacted with glycerol carbonate and at least one other cyclic carbonate, particularly in the presence of a base catalyst.

The particular type of copolymeric product can readily be determined by controlling how the carbonate reagents are supplied to the reaction. Thus, random (statistical) copolymers can be made by supplying a mixture of carbonate reagents to the reaction; block copolymers by substantially completing reaction with one carbonate before the (an)other is added; taper block copolymers by adding the (an)other carbonate reagent later than but before complete reaction of a first carbonate reagent. Sequential block, block random and similar types of copolymeric chains can be made by combinations or ready variations on the above reaction sequences.

In addition to the compounds of the invention, typical synthesis reactions may generate (poly)glycerol in a side reaction by polymerisation of glycerol carbonate onto the free OH group of glycerol carbonate. Generally, the more glycerol carbonate (as such) present in the reaction system the more likely polyglycerol is to be made and consequently, aliquot or gradual addition of glycerol carbonate over the course of the reaction reduces the amount of polyglycerol made.

It may be desirable to include reducing agent in the reaction to aid in colour control, particularly as thermal exposure of sorbitan esters, especially unsaturated fatty acid sorbitan esters, may give rise to more highly coloured products. Reducing agents commonly used for this purpose, particularly in the manufacture of food or personal care products, can be used in this invention and examples include phosphorous acid (H₃PO₃), hypophosphorous acid (H₃PO₂) and borohydride (usually as sodium borohydride). Where the reducing agent is itself an acid e.g. phosphorous or hypophosphorous acid, it will usually be present as a salt, typically an alkali metal salt. The salt may be made in situ by reaction with base e.g. part of the basic catalyst (where used) and in this case care may be needed to ensure that sufficient base is present to neutralise the reducing acid and to act as catalyst. When used the amount of reducing agent will typically be from 0.1 to 15%, more usually 1 to 10%, and particularly 2 to 7.5%, by mole based on the sorbitan ester starting material.

Another way of reducing product colour is to include particulate carbon, particularly so-called “activated carbon”, or a bleaching earth e.g. diatomaceous earth, in the reaction to absorb coloured side products. When used, the amount of carbon will typically be from 0.5 to 2.5 weight % of the total reagents. Of course, this carbon or bleaching earth will generally be removed e.g. by filtration, before the products are included in end use formulations. Activated carbon and a reducing agent may be used together in the reaction if desired.

Further colour improvement can be achieved by treatment of the reaction product with particulate carbon, particularly activated carbon, or bleaching earth, typically at from 0.5 to 2.5 weight % of the product, or by bleaching the product of the reaction e.g. with a peroxide based bleach, generally after removal of any activated carbon or bleaching earth.

Typically the reaction temperature will be superambient, typically at least 100° C. and more usually at least 170° C. and can range up to 250° C., with the range 180 to 240° C. being generally suitable. We have found that it is usually desirable to use somewhat higher reaction temperatures with relatively longer chain carboxylic acid sorbitan esters to counteract the trend towards reduced compatibility of the sorbitan ester with the glycerol carbonate. Such reduced compatibility would otherwise tend to lead to possible phase separation and, by thus slowing the desired reaction, increased production of polyglycerol by-product.

The reaction and its completion can conveniently be monitored using standard IR e.g. FT-IR, and HPLC techniques. Under the conditions set out above the reaction generally runs to completion (monitored as described above) so that the reaction mixture is the sorbitan ester polyglycerol ether product together with catalyst residues and, generally low levels of, impurities (other than polyglycerol—see discussion above). We have seen reaction times typically in the range 1 to 20 hours with most being complete in from 1.5 to 15 hours, usually from 2 to 7 hours. In practice additional time under reaction conditions may be used to ensure complete reaction.

We have found that typically the reactions to make the compounds of the invention can be carried out without the need for a solvent or diluent and we expect that this is how the reaction will be carried out generally, particularly as this will avoid any problem in isolating the desired product. However a suitable inert reaction medium, solvent or diluent may be used if desired. Suitable such materials are liquids which remain thermally stable and are inert to the reagents and products. Any solvent used will either have a relatively low vapour pressure at the reaction temperature or the reaction will be conducted under suitable containment or reflux arrangements. Suitable examples of solvents or diluents include dimethyl iso-sorbide (BP 118 to 120° C. at 20 mbar), dimethylformamide (BP 153° C.), dimethylsulfoxide (BP 189° C.), ethylene glycol and diethylene glycol diethers e.g. dimethyl, diethyl or dibutyl ethers.

Solvent and/or diluent may be included with the product, either by leaving reaction solvent/diluent in the product or by subsequent addition, to reduce product viscosity for transport, storage and/or subsequent use. Suitable solvents/diluents for this purpose include those mentioned above as well as glycerol carbonate (when its reactivity does not interfere with downstream product use), glycerol or, and particularly, monopropylene glycol because this may give the additional benefit of improving the molecular packing of the polyglycerol ether products at the phase interface in end use formulations. Typically such solvents/diluents will be used in amounts to give formulations having from 50 to 90, more usually 60 to 80 and particularly about 70, % by weight of the polyglycerol ether product.

Typically, the reagents used to make the compounds of the invention remain liquids of low vapour pressure at reaction temperatures so the reaction can be conveniently carried out at ambient pressure though moderately superambient pressure may be used if desired. We think it unlikely that it will be desirable to use subambient pressure but by choosing suitable involatile reagents it may be possible to carry the reaction out at moderately subambient pressure.

To help avoid excessive colour generation, particularly when reacting unsaturated acid sorbitan esters, the synthesis reactions will usually be carried out in a largely oxygen free atmosphere, e.g. in a nitrogen atmosphere. In laboratory scale synthesis, this has not needed to be more elaborate than using a nitrogen blanket or sparge. Larger scale manufacture may be less sensitive because of the relatively lower exposed surface area generally possible in such larger scale synthesis.

Generally we expect that synthesis reactions will be carried out in a batch mode, typically by mixing the reagents in a suitable vessel and allowing them to react, usually under stirring for a suitable time (see above). As noted above fresh reagent, particularly glycerol carbonate, and/or catalyst may be added occasionally, at multiple intervals or continuously during the reaction (semi-batch operation). It is also possible to use continuous or semi-continuous reaction modes if desired.

The compounds of the invention can be used in a wide variety of applications. In food and/or cosmetic applications and products, they are typically used as oil in water and sometimes as water in oil emulsifiers, solubilizers, emollients, dispersants, spreading agents and rheology modifiers. In industrial applications, they are used as oil in water and sometimes as water in oil emulsifiers, dispersants, and potentially in antifog, antistatic, lubrication or plasticizer applications.

The invention accordingly includes an emulsion, particularly an oil in water or water in oil emulsion, which is emulsified with or stabilised by a compound of the invention.

The following Examples illustrate the invention. All parts and percentages are by weight unless otherwise stated.

### Materials

### Sorbitan Esters

Est1 sorbitan monolaurate; Span 20 ex Croda
Est2 sorbitan monopalmitate; Span 40 ex Croda
Est3 sorbitan monostearate; Span 60 ex Croda
Est4 sorbitan monooleate; Span 80 ex Croda
Est5 1:1 (molar) mixture of Est1 and Est3
Comp1 sorbitan monolaurate 20E0; Tween 20 ex Croda
Comp2 sorbitan monooleate 20E0; Tween 80 ex Croda

### Catalysts

### Cat1 NaOH

### Cat2 K₂CO₃

Cat3 NaOH plus H₃PO₃ at a weight ratio 1:0.88; molar ratio 2.33:1

### Oils

Oil1 hexadecane Arlamol HD ex Croda

### Test Methods

Surface Tension (ST) was measured on a 0.01% solution of test sample in demineralised water using a Kruss Digital Tensiometer at 26° C. and the results given in mN.m⁻¹ (=dyne.cm⁻¹).
Emulsion Stability—was assessed by making up oil in water emulsions as described below. 100 g of formulation was made by dissolving 1 g of test product in 79 g distilled water and heating to 75° C. in a water bath. 20 g of Oil1 was separately heated to 75° C. on a water bath and at 75° C., the oil phase was added to the water phase under stirring with an overhead driven propeller blade stirrer [500 rpm (ca 8 Hz)] and then homogenised with an Ultra Turrax [12000 rpm (200 Hz)] for 2 minutes. The emulsion was then allowed to cool to ambient temperature under stirring [overhead stirrer at 300 rpm (5 Hz)]. Generally, the test system separates into oil rich (upper) and oil lean (lower) layers and testing data relates to the oil rich upper layer. Emulsions were stored at ambient temperature and at 50° C. and the emulsion stability was visually assessed at intervals of 1 day (1 d), 1 week (1 w) and 1 month (1 m). The emulsions were rated as: NS=no separation; TTO=trace of oil separation at the top of emulsion; x %=percent oil separation at top of emulsion; Br=emulsion broken.

### SYNTHESIS EXAMPLES

### Synthesis Example

### SE1

Sorbitan ester, Est1 (25.95 g; 0.075 mol), glycerol carbonate (35.4 g; 0.3 mol) and sodium hydroxide (0.15 g; 5 mol % based on Est1), were charged to a 100 ml round bottomed flask fitted with magnetic stirrer bar, nitrogen sparge, side-arm water cooled condenser and collection flask.

The mixture was heated under stirring and gentle nitrogen sparge on an oil bath, itself heated with a hotplate with stirrer motor until the oil temperature was 190° C. The reaction mixture was then maintained at an oil bath temperature of 190° C., until all of the glycerol carbonate had been consumed, as monitored by FT-IR. The reaction was then stopped and the product discharged.

### Synthesis Examples

### SE2 to SE12

Further polyglycerol ethers of sorbitan fatty acid esters were made by the general method set out in Synthesis Example SE1 making appropriate changes to the materials, proportions or conditions. The reactions were monitored and the identity of the products was confirmed using IR spectroscopy and HPLC. The materials used (GC=glycerol carbonate), reaction conditions (React Conds) and products (including SE1) are summarised in Table SE 1 below.

### Synthesis Example SE13

A mixture of Est1 (51.9 g; 150 mmol), glycerol carbonate (17.7 g; 150 mmol) (1 mole per mole of Est1) and catalyst Cat3 (0.47 g) (0.9 wt % catalyst based on Est1) was slowly heated to 210° C. on an oil bath under N₂ sparge. After stirring at this temperature for 30 min the remainder of the glycerol carbonate (247.8 g; 2100 mmol; 14 moles per mole of Est 1) was added slowly using a peristaltic pump over a period of 3.5 hours (4 moles per hour). After completing the addition, the reaction mixture was stirred at 210° C. for a further 1 hour to ensure complete reaction (confirmed by IR spectroscopy showing the absence of glycerol carbonate) and then cooled and discharged. The materials used, reaction conditions and products are summarised in Table SE 1 below.

TABLE SE1

| SE | | GC | Catalyst | | React Conds | | ST |
|---|---|---|---|---|---|---|---|
| No | Ester | (mol) | type | mol % | time (hrs) | temp (° C.) | (mN · m⁻¹) |
| SE1 | Est1 | 4 | Cat1 | 5 | 4 | 190 | 36.2 |
| SE2 | Est1 | 10 | Cat1 | 5 | 6.5 | 190 | 35.0 |
| SE3 | Est1 | 15 | Cat1 | 5 | 3.5 + 4.5 | 180 + 190 | 37.9 |
| SE4 | Est1 | 20 | Cat1 | 5 | 7 | 200 | 36.1 |
| SE5 | Est2 | 10 | Cat2 | 10 | 2.5 | 210 | 41.1 |
| SE6 | Est2 | 20 | Cat2 | 10 | 2 | 220 | 41.4 |
| SE7 | Est3 | 10 | Cat2 | 10 | 2 | 220 | 45.7 |
| SE8 | Est3 | 20 | Cat2 | 10 | 3.5 | 220 | 46.7 |
| SE9 | Est4 | 10 | Cat1 | 5 | 5 | 210 | 40.0 |
| SE10 | Est4 | 20 | Cat2 | 10 | 3 | 230 | 35.7 |
| SE11 | Est4 | 30 | Cat2 | 10 | 2.5 + 2.5 | 220 + 235 | 38.0 |
| SE12 | Est5 | 10 | Cat2 | 5 | 2 | 210 | 35.1 |
| SE13 | Est1 | 15 | Cat3 | (see text) | 5 | 210 | — |

### Application Examples

Some of the polyglycerol esters made in Synthesis Examples SE1 to SE12 were screened for their ability to stabilise oil in water emulsions as described above. The results are set out in Table AE1 below (Mol ratio is GC:sorbitan ester in synthesis).

TABLE AE1

| Ex | SE | | NaCl | Amb | | | 50° C. | | |
|---|---|---|---|---|---|---|---|---|---|
| No | No | Mol ratio | (wt %) | 1 d | 1 w | 1 m | 1 d | 1 w | 1 m |
| AE1Ca | Comp1 | N/A | 0 | NS | NS | NS | NS | NS | TTO |
| AE1Cb | | | 5 | NS | NS | NS | NS | NS | TTO |
| AE1.1 | SE1 | 1:4 | 0 | NS | NS | NS | NS | TTO | 5% |
| AE1.1a | | | 5 | 5% | Br | — | Br | — | — |
| AE1.2 | SE2 | 1:10 | 0 | NS | NS | NS | NS | NS | TTO |
| AE1.2a | | | 5 | TTO | 5% | 10% | TTO | TTO | TTO |
| AE1.3 | SE3 | 1:15 | 0 | NS | NS | NS | NS | NS | TTO |
| AE1.3a | | | 5 | NS | TTO | TTO | NS | TTO | TTO |
| AE1.4 | SE4 | 1:20 | 0 | NS | NS | NS | NS | NS | NS |
| AE1.4a | | | 5 | NS | TTO | TTO | NS | TTO | TTO |
| AE1.5 | SE5 | 1:10 | 0 | NS | NS | NS | NS | NS | NS |
| AE1.5a | | | 5 | TTO | TTO | TTO | TTO | Br | — |
| AE1.6 | SE7 | 1:10 | 0 | NS | NS | NS | NS | NS | TTO |
| AE1.6a | | | 5 | NS | TTO | TTO | TTO | Br | — |
| AE1.7 | SE10 | 1:20 | 0 | NS | NS | NS | NS | NS | TTO |
| AE1.7a | | | 5 | TTO | 5% | 10% | 10% | 15% | 15% |
| AE2Ca | Comp2 | N/A | 0 | NS | NS | NS | NS | TTO | TTO |
| AE2Cb | | | 5 | TTO | TTO | TTO | TTO | TTO | TTO |

1. A compound which is a polyglycerol ether of a sorbitan carboxylic acid ester.

2. A compound as claimed in claim 1 which is a polyglycerol ether of a C₈ to C₂₂, carboxylic acid sorbitan monoester.

3. A compound as claimed in claim 1 of the formula (I):
Sor(R¹)(R²)(R³)(R⁴) (I)
where
Sor is a sorbitan residue;
one of R¹, R², R³ and R⁴ is a group of the formula (II): —O₂CR⁵ where R⁵ is a C₇ to C₂₁ hydrocarbyl group;
one of R¹, R², R³ and R⁴ is a group of the formula (III): -[Gly]ₙ-[AO]ₘ-H where Gly is a glycerol residue; AO is an alkyleneoxy residue of a corresponding diol cyclic carbonate, in any order; n is an average of from 0 to 100; and m is an average of from 0 to 75;
each of the remaining two of R¹, R², R³ and R⁴ is independently:
a group of the formula (IIa): —O₂CR^{5′} where R^{5′} is a C₁ to C₂₁ hydrocarbyl group; or
a group of the formula (III): -[Gly]ₙ[AO]ₘ-H where each Gly, AO, n and m is independently as defined above;
such that the total of all the indices n is at least 1.

4. A compound as claimed in claim 3 where one of the groups R¹, R², R³ and R⁴ is a group —O₂CR⁵ and the remaining three groups are of the formula (III): -[Gly]ₙ-[AO]ₘ-H where R⁵, Gly, AO, n and m are as defined in claim 3.

5. A compound as claimed in claim 1 which is a mixed poly(alkyleneoxy)/polyglycerol ether of a sorbitan carboxylic acid ester.

6. A compound as claimed in claim 5 of the formula (Ia):
Sor(R^{1a})(R^{2a})(R^{3a})(R^{4a}) (Ia)
where
Sor is a sorbitan residue;
one of R^{1a}, R^{2a}, R^{3a} and R^{4a} is a group of the formula (II): —O₂CR⁵ where R⁵ is a C₇ to C₂₁ hydrocarbyl group;
one of R^{1a}, R^{2a}, R^{3a} and R^{4a} is a group of the formula (IIIa): -[Gly]_{n′}-[AO]_{m′}-H where Gly is a glycerol residue and AO is an alkyleneoxy residue of a corresponding diol cyclic carbonate, in any order; n′ is an average of from 0 to 100; and m′ is an average of from 0 to 75;
each of the remaining two of R^{1a}, R^{2a}, R^{3a} and R^{4a} is independently a group of the formula (IIa): —O₂CR^{5′} where R^{5′} is a C₁ to C₂₁ hydrocarbyl group; or
a group of the formula (IIIa): -[Gly]_{n′}-[AO]_{m′}-H where Gly, AO, n′ and m′ are as defined above;
such that the total of all indices n′ is at least 1 and the total of all indices m′ is at least 0.1.

7. A compound as claimed in claim 1 where the average degree of polymerisation is from 5 to 75, particularly from 10 to 50.

8. A method of making a polyglycerol ether of a sorbitan carboxylic acid ester which comprises reacting a sorbitan ester with at least 1, and desirably at least 3, moles of glycerol carbonate per mole of sorbitan ester.

9. A method as claimed in claim 8 wherein the sorbitan ester is a C₈ to C₂₂, particularly a C₁₀ to C₂₂, and especially a C₁₂ to C₁₈, carboxylic acid sorbitan monoester.

10. A method as claimed in claim 8 which comprises reacting a sorbitan ester of the formula (IV):
Sor(R^{1′})(R^{2′})(R^{3′})(R^{4′}) (IV)
where
Sor is a sorbitan residue;
one of R¹′, R²′, R³′ and R⁴′ is a group of the formula (II): —O₂CR⁵ where R⁵ is a C₇ to C₂₁ hydrocarbyl group;
one of R¹, R^{2′}, R^{3′} and R^{4′} is a hydroxyl group; and
each of the remaining two of R¹, R², R³ and R⁴ is independently a hydroxyl group, or
a group of the formula (IIa): —O₂CR^{5′} where R^{5′} is a C₁ to C₂₁ hydrocarbyl group;
with at least 1 mole of glycerol carbonate per mole of sorbitan ester of the formula (IV).

11. A method as claimed in claim 8 which comprises reacting a sorbitan ester with glycerol carbonate and at least one other cyclic carbonate.

12. A method as claimed in claim 11 wherein the other cyclic carbonate is ethylene glycol carbonate, propylene glycol carbonate and/or propylene1,3-diol (trimethylene)carbonate.

13. A method as claimed in claim 8 wherein the molar ratio of sorbitan ester to glycerol carbonate is from 1:3 to 1:100, more usually from 1:3 to 1:75, desirably from 1:3 to 1:50, more usually from 1:3 to 1:40 and particularly from 1:3 to 1:30.

14. A method as claimed in claim 8 wherein the reaction mixture includes a catalyst.

15. A method as claimed in claim 14 wherein the catalyst is a basic catalyst.

16. A method as claimed in claim 15 wherein the catalyst is at least one alkali metal hydroxide, carbonate or alkoxide and/or at least one tertiary amine.

17. A method as claimed in claim 14 wherein the amount of catalyst is from 0.5 to 25%, more usually 2 to 20%, and particularly 5 to 15%, by mole based on the sorbitan ester starting material.

18. A method as claimed in claim 8 wherein the reaction mixture further includes a reducing agent and/or activated carbon and/or bleaching earth and/or the reaction product is treated with activated carbon and/or a bleaching agent.

19. A method as claimed in claim 18 wherein the reducing agent is at least one of phosphorous acid, hypophosphorous acid and borohydride.

20. A method as claimed in claim 18 wherein the amount of reducing agent is from 0.1 to 15%, more usually 0.2 to 10%, and particularly 21 to 7.5%, by mole based on the sorbitan ester starting material.

21. A method as claimed in claim 8 wherein the reaction temperature is from 100° C. to 250° C., suitably 180 to 220° C.

22. A method as claimed in claim 8 wherein the reaction is carried out in an inert, particularly a nitrogen, atmosphere.

23. A method as claimed in claim 8 wherein the reaction is carried out in an inert solvent or diluent.

24. An emulsion, particularly an oil in water or water in oil emulsion, which is emulsified with or stabilised by a compound, which is a polyglycerol ether of a sorbitan carboxylic acid ester, or made by the method of claim 9.
